(19) United States
(12) Patent Application Publication
(10) Pub. No.: US 2008/0287897 A1
(43) Pub. Date: Nov. 20, 2008

(54) Absorbent article and method of making same

(75) Inventors: Loida Guzman Reyes, Philadelphia, PA (US); Brian A. McGinty, Collegeville, PA (US); Ron Rothenberger, Collegeville, PA (US); Linda Morrell-Schwartz, Bensalem, PA (US); Edward P. Erdman, West Chester, PA (US); Andrew Waksmundzki, Jackson, NJ (US)

Correspondence Address: RATNERPRESTIA, P O BOX 980, VALLEY FORGE, PA, 19482-0980, US

(73) Assignee: Tyco Healthcare Retail Services AG

(21) Appl. No.: 11/904,467

(22) Filed: Sep. 27, 2007

Related U.S. Application Data
(60) Provisional application No. 60/930,117, filed on May 14, 2007.

(51) Int. Cl. A61F 13/15

(52) U.S. Cl. 604/365; 604/385.01; 604/385.22; 604/385.27

(57) ABSTRACT

Disposable protective undergarments and methods for manufacturing the same, in which the undergarments include a front portion and a back portion each having a stretchable waist area, a stretchable leg area longitudinally spaced from the waist area, and a stretchable belly area positioned between the waist area and the leg area is provided. The belly area is separated from the waist area thereby defining a substantially non-stretchable portion therebetween, and the belly area is also separated from the leg area thereby also defining a substantially non-stretchable portion therebetween. The undergarment optionally includes front and back portions with waist areas each having an outer layer folded over an inner layer, between which layers stretchable web material is sandwiched between.

### CROSS REFERENCE TO RELATED APPLICATIONS

This application claims priority to U.S. Provisional Patent Application Ser. No. 60/930,119, filed May 14, 2007, which is incorporated herein by reference.

### FIELD OF THE INVENTION

This invention relates generally to disposable absorbent undergarments, e.g., disposable underwear, adult and youth briefs, and training pants for children. More specifically, this invention relates to articles of the type that can be manufactured efficiently and economically and which exhibit protection from leakage, good fit and comfort.

### BACKGROUND OF THE INVENTION

Absorbent articles such as disposable diapers, training pants, adult incontinence garments, and the like are known, their major function being to absorb and contain body exudates. Such articles are thus intended to prevent the soiling, wetting, or other contamination of clothing or other articles, such as bedding, that come into contact with the wearer. In the case of disposable protective undergarments, a variety of designs and manufacturing techniques have been developed to maximize performance and comfort to the wearer and to obtain a high degree of efficiency in manufacturing while minimizing the amount of waste material, thereby keeping production costs as low as possible. However, many of the current designs and manufacturing methods have been unable to achieve an optimum balance between these desirable, yet often diverging goals. While many developments have been made in the art of disposable absorbent undergarments, there remains a need for further improvements in both design and manufacturing.

### SUMMARY OF THE INVENTION

The invention is directed to disposable protective undergarments. In one aspect of the invention, the invention provides a protective undergarment comprising a waist opening, a front portion and a back portion. Each of the front and back portions comprises a non-woven layer having a leg area, a belly area and a waist area, with the non-woven layer extending proximal to the waist opening, and a stretch material coupled to the non-woven layer and extending about the waist opening. The non-woven layer is folded over the stretch material at the waist opening to capture the stretch material between portions of the non-woven layer.

In another aspect, the invention provides a protective undergarment comprising a front portion and a back portion, each portion comprising an outer layer and an inner layer having different lengths. Each of the front and back portions have a waist area comprising a stretchable web having a width to thickness ratio of at least 3:1, a leg area longitudinally spaced from the waist area, and a belly area positioned between the waist area and the leg area. The longer of the inner and outer layers of the front and back portions is folded over the shorter of the inner and the outer layers of the front and back portions at the waist end to sandwich the stretchable web therebetween.

In yet another aspect, the invention provides a protective undergarment comprising a front portion and a back portion each having a stretchable waist area, a stretchable leg area longitudinally spaced from the waist area, and a stretchable belly area positioned between the waist area and the leg area. The belly area is separated from the waist area thereby defining a substantially non-stretchable portion therebetween, and the belly area is also separated from the leg area thereby also defining a substantially non-stretchable portion therebetween.

In still yet another aspect, the invention provides a method of manufacturing a protective undergarment comprising the step of forming from at least one continuous web of non-woven material front and back outer portions. The method further includes the steps of coupling a first continuous web of stretch material to the front outer portion of the non-woven material to form a front waistband and coupling a second continuous web of stretch material to the back outer portion of the non-woven material to form a rear waistband. The method also includes the steps of folding a portion of said non-woven material over the front and rear waistbands, and connecting the front and rear waist bands to form a continuous waist opening.

In yet still another aspect, the invention provides a method of manufacturing a protective undergarment comprising the steps of forming from at least one continuous web of non-woven material front and back outer portions. The method further includes the steps of coupling a first continuous web of stretch material having a width to thickness ratio of at least 3:1 to the front outer portion of the non-woven material to form a front waistband and coupling a second continuous web of stretch material having a width to thickness ratio of at least 3:1 to the back outer portion of the non-woven material to form a rear waistband. The method also includes the step of connecting the front and rear waist bands to form a continuous waist opening.

### BRIEF DESCRIPTION OF THE DRAWINGS

The invention is best understood from the following detailed description when read in connection with the accompanying drawing. It is emphasized that, according to common practice, the various features of the drawing are not rendered to scale. On the contrary, the dimensions of the various features are arbitrarily expanded or reduced for clarity. Included in the drawing are the following figures, in which like reference numerals refer to similar features in the respective Figures:

FIG. 1 is a plan view of an exemplary embodiment of a disposable absorbent undergarment in a partially unassembled, flat state in which the body-side surface of the absorbent undergarment faces the viewer.

FIG. 2 is a back view of the absorbent undergarment of FIG. 1 in its assembled state.

FIG. 3 is a partial side cross-sectional view of the front and back portions of the absorbent protective undergarment as shown in FIG. 1.

FIG. 4 is an exploded side cross-sectional view of the absorbent composite of the absorbent protective undergarment of FIG. 1, according to one aspect of this invention.

FIG. 5 is an illustration showing the front and back portions of the absorbent undergarment of FIG. 1 at a step in the manufacturing process before the portions have been separated from one another and shifted so as to be position for receiving a crotch insert.

FIG. 6 is a plan view of another exemplary embodiment of an absorbent undergarment in a partially unassembled, flat state in which the body-side surface of the absorbent undergarment faces the viewer.

FIG. 7 is a back view of the absorbent undergarment of FIG. 6 in its assembled state.

FIG. 8A is a partial side cross-sectional view of one embodiment of the back portion of the absorbent undergarment of FIG. 6.

FIG. 8B is a partial side cross-sectional view of another embodiment of the back portion of the absorbent undergarment of FIG. 6.

FIG. 9 is back view of another exemplary embodiment of an absorbent undergarment in its assembled state illustrating an exemplary ultrasonic bond pattern.

FIG. 10 is a back view of another exemplary embodiment of an absorbent undergarment in its assembled state illustrating another exemplary ultrasonic bond pattern.

FIGS. 11A through 11E are illustrations of exemplary ultrasonic bond patterns that can be applied to the absorbent undergarment according to an embodiment of the invention, for example as shown in FIGS. 8 and/or 9.

FIGS. 12A and 12B are plan views of alternative embodiments of the invention illustrating exemplary front and back portions before and after separation, illustrating exemplary wave-shaped cuts for producing a wave-shaped pattern along a crotch end of the front and back portions.

FIGS. 13A and 13B are plan views of alternative embodiments of the invention illustrating exemplary front and back portions before and after separation and assembly with a crotch insert, illustrating exemplary wave-shaped cuts for producing a wave-shaped pattern along a crotch end of the front and back portions.

FIG. 14 is a plan view of yet another exemplary embodiment of a disposable absorbent undergarment in a partially unassembled, flat state in which the body-side surface of the absorbent undergarment faces the viewer.

FIG. 15 is a side cross-sectional view of the front and back portions of the absorbent protective undergarment as shown in FIG. 14.

FIG. 16 is a simplified schematic plan view of still yet another exemplary embodiment of the invention illustrating the assembly of the crotch insert to the front and back portions in which the body-side of the absorbent undergarment faces the viewer.

FIG. 17 is a side cross-sectional view of the front and back portions of the absorbent protective undergarment of FIG. 15, shown as modified according to FIG. 16.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

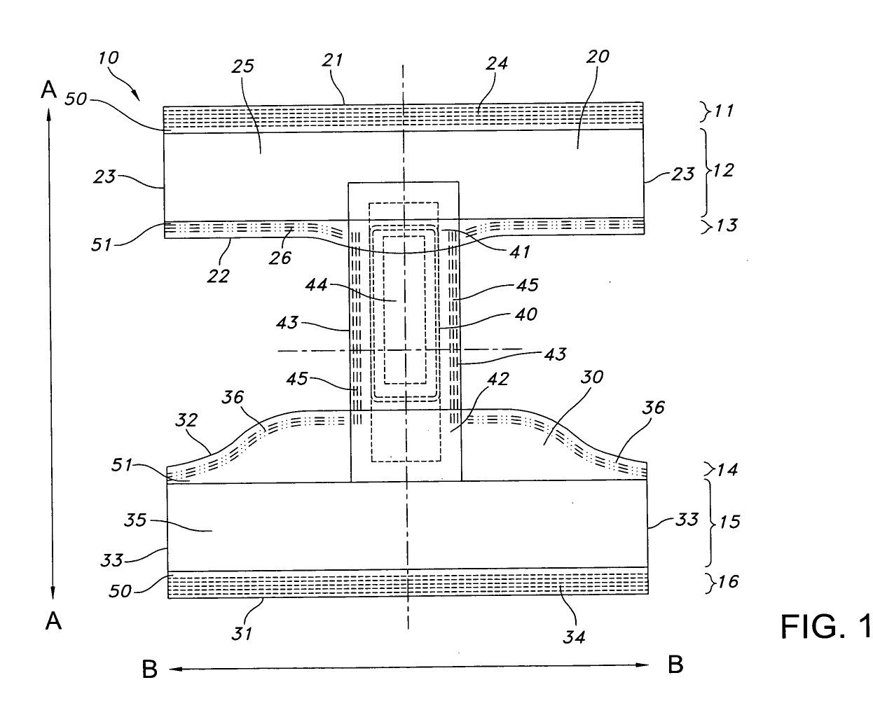

Referring generally to the drawing, FIG. 1 illustrates an embodiment in which the present invention is shown in a partially unassembled, flat state, the body-side surface of the disposable absorbent undergarment 10 facing the viewer. The arrows A-A generally indicate a longitudinal dimension of the undergarment 10 in the partially unassembled state. This longitudinal orientation corresponds to the longitudinal orientation of the crotch insert 40 of the undergarment 10, as will be described later. Also, the arrows B-B generally indicate the width dimension of the undergarment 10 in the partially unassembled state. The direction B-B also corresponds to the machine direction optionally used to make at least some of the components of the undergarment 10. More specifically, front 20 and back 30 portions of the undergarment 10 (described below) are optionally formed from a continuous web or continuous webs that are directed along a machine direction parallel to B-B.

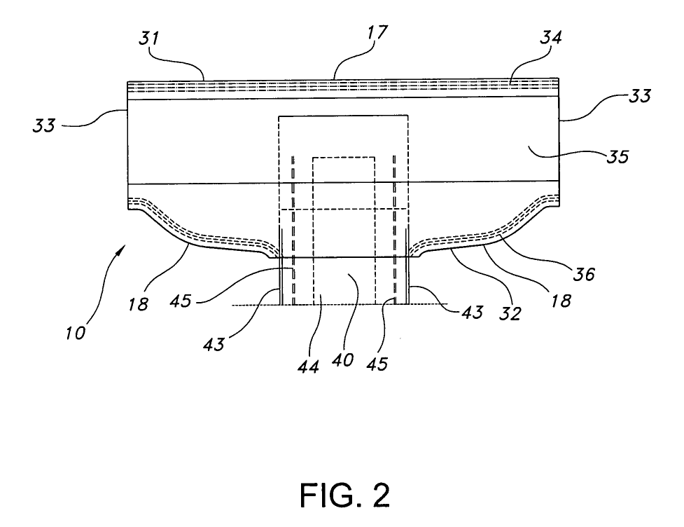

With reference to FIG. 1, the disposable protective undergarment 10 is provided comprising a front portion 20, a back portion 30, and a crotch insert 40. Fully assembled, as shown in FIG. 2, the disposable absorbent undergarment 10 forms a pants-shaped chassis. As shown in its assembled configuration, the undergarment includes waist opening at 17 and leg openings at 18 located on opposing sides of the crotch insert 40.

As shown in FIG. 1, an exemplary configuration of the disposable protective undergarment 10 comprises a front portion 20 including a substantially straight waist end 21 along a transverse direction, a crotch end 22 longitudinally spaced from the waist end 21 having a wave-shaped pattern running in a transverse direction, and two substantially straight sides 23 that extend between the waist end 21 and the crotch end 22. Again, the term “longitudinally” as used herein refers to the direction along line A-A, while the term “transverse” refers to the direction along line B-B, as shown in FIG. 1. The front portion 20, as shown in FIG. 1, includes a waist area 11, a belly area 12, and a leg area 13.

Similar to the front portion 20, the back portion 30 of the disposable protective undergarment 10, according to the exemplary embodiment of FIG. 1, includes a substantially straight waist end 31 along a transverse direction, a crotch end 32 longitudinally spaced from the waist end 31 and extending in a transverse direction, and two substantially straight sides 33 extending between the waist end 31 and the crotch end 32. The crotch end 32 of the back portion 30 has a wave-shaped pattern along its transverse length, and further includes a substantially straight crotch end portion where the crotch insert 40 is fixed to the back portion 30. The back portion 30 is separated longitudinally from the front portion 20 by a gap between the two portions. The back portion 30, as shown in FIG. 1, includes waist area 16, mid-section 15, and leg area 14. For simplicity, mid-section 15 will hereinafter be referred to as belly area 15.

As illustrated in FIG. 1, crotch insert 40 has a front end portion 41, a back end portion 42 longitudinally spaced from the front end 41, and two sides 43 extending between the front end portion 41 and the back end portion 42. The front end portion 41 is fixed to front portion 20 and the back end portion 42 is fixed to back portion 30 thereby bridging the gap between the crotch end 22 of the front portion 20 and the crotch end 32 of the back portion 30. Further, the crotch insert 40, as shown in FIG. 1, has a generally rectangular shape. The crotch insert 40 may also have other shapes, for example an oval shape or any other straight-sided or curved shape. In an embodiment of the invention, the crotch insert 40 may include an absorbent composite 44 attached to or otherwise associated with a central portion of the crotch insert 40.

When fully assembled, as shown in FIG. 2, the sides 23 of the front portion 20 are fixed to the respective sides 33 of the back portion 30 so as to form a continuous waist comprising the waist end 21 of the front portion and the waist end 31 of the back portion. Further, the crotch end 22 of the front portion 20 and the crotch end 32 of the back portion 30, along with the sides 43 of the crotch insert together define leg openings 18. As shown in FIG. 1, each leg opening 18 may have a wave-shaped edge at the crotch end 22 of the front portion 20; a wave-shaped edge at the crotch end 32 of the back portion 30; and an intermediate, substantially straight edge along the sides 43 of the crotch insert 40.

Referring again to the exemplary embodiment of the invention illustrated in FIG. 1, the front portion 20 includes waist elastics 24 in waist area 11, belly elastic(s) 25 in belly area 12, and leg elastics 26 in leg area 13. Similarly, the back portion 30 includes waist elastics 34 in waist area 16, belly elastic(s) 35 in belly area 15, and leg elastics 36 in leg area 14. Each of the waist elastics 24, 34, belly elastic(s) 25, 35, and leg elastics 26, 36 extend along the entire transverse length of the front portion 20 and back portion 30. In this embodiment, the waist elastics 24, 34 and belly elastic(s) 25, 35 are substantially parallel to the waist ends 21, 31 of the undergarment 10. The leg elastics 26, 36, however, correspond to the profile of the crotch edge 22 of the front portion 20 and the crotch edge 32 of the back portion 30.

According to the embodiment as shown in FIG. 1, the invention includes leg elastics 26, 36 that assist in preventing leakage on the outer part of a wearer's leg, by conforming to the shape of the front and back portions of the wearer and giving the appearance of regular underwear. As shown in FIG. 1, to obtain a better fit, the leg elastic contains a wave-cut profile that substantially matches the contours around the front thigh of the wearer. For example, the front wave-cut is substantially straight at the sides, but has its highest amplitude where the front panel 20 is attached to the crotch insert 40. Alternatively, the undergarment 10 may have a leg profile design on the front and back. Further, for better fit, the undergarment 10 includes crotch insert leg elastics 45 that are straight and aligned to be parallel to the sides 43 of the crotch insert 40. Other designs and configurations, however, may also be incorporated.

In addition, as shown in FIG. 1, portions of the belly elastics 25, 35 and/or the leg elastics 26, 36 may be deactivated where the crotch insert 40 overlaps the front 20 and back 30 portions. In other words, the elastics 25, 35 and 26, 36 may lose substantial stretchability in the transverse direction when overlapped by the crotch insert 40, which is not substantially stretchable in the transverse direction. Alternatively, the elastics may be deactivated by the removal of or a cutting of a portion of the elastics at the location where the crotch insert 40 is attached to each of the front 20 and back 30 portions.

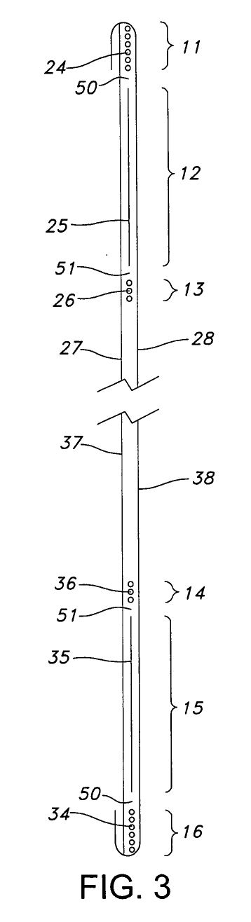

Referring to FIG. 3, which is a side cross-sectional view of the front portion 20 and back portion 30 of FIG. 1, the undergarment 10 includes a front outer portion layer 28 folded over waist elastics 24 and the front inner portion layer 27. Similarly, the back outer portion layer 38 is folded over waist elastics 34 and the back inner portion layer 37. Interposed between the front and back outer portion layers 28, 38 and inner portion layers 27, 37, the undergarment 10 includes stretchable waist areas 11, 16, stretchable belly areas 12, 15, and stretchable leg areas 13, 14. The stretchable waist areas 11, 16 include waist elastics 34, such as elastic threads or filaments. The stretchable belly areas 12, 15 include belly elastics 25, 35, shown as elastomeric film. And the stretchable leg areas 13, 14 include leg elastics 26, 36, which have elastic threads or filaments. Thus, as can be seen from FIG. 3, the front portion 20 and back portion 30 of the undergarment 10 are essentially mirror images of one another. Suitable elastomeric films that may be used in the present invention preferably include, bi-directional film manufactured by Pliant Corp. Suitable elastic threads or filaments that may be used in the present invention include, for example, LYCRA® manufactured by INVISTA, or CREORA® spandex (or elastane) manufactured by Hyosung, Inc.

The material forming the front 20 and rear 30 portions of the chassis, including the back portion inner and outer layers 37, 38 and front inner and outer layers 27, 28, shown in FIG. 3, can be any suitable material used in disposable absorbent articles. In the interest of wearing comfort, the materials are preferably non-woven, cloth-like breathable materials, such as spunbond/meltblown/spunbond polypropylene at 15 gsm available from Avgol of Holon, Israel, for example. The material making up the chassis may be a polylaminate, e.g., polyfilm bonded to a non-woven material. The adhesive for securing the webs together with the elastic materials interposed therebetween, may be a construction adhesive, such as that available from National Starch and Chemical Company of Bridgewater, N.J. The product will use soft stretch or non-stretch materials (such as Av-soft from Avgol). Alternatively, the webs may be secured together using ultrasonic bonds or other securement means.

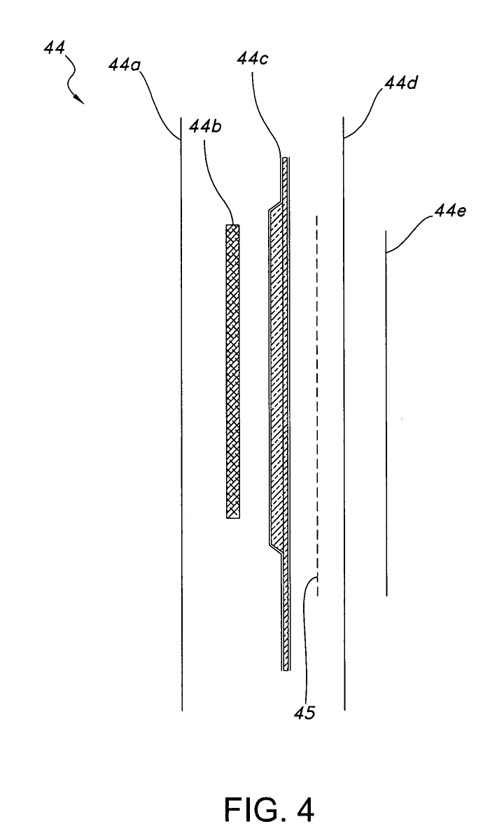

The absorbent composite of an exemplary embodiment of the disposable absorbent undergarment can be configured as shown in FIG. 4. This embodiment includes a multilayer absorbent composite 44 having a body side (left side as shown in FIG. 4) and a garment side (right side as shown in FIG. 4). As shown in the cross-sectional view, beginning from body-side and continuing to the garment side, the absorbent composite 44 includes a cover 44a adjacent an acquisition/distribution layer (ADL) 44b, which is disposed adjacent an absorbent core 44c, which is adjacent a barrier layer 44d. Adjacent the body-side of the backsheet are disposed an insert outer cover, or backsheet, 44e. Crotch insert elastics 45 are disposed between cover 44a and barrier layer 44d.

In another embodiment, the ADL may comprise two separate layers including an acquisition layer and a distribution layer. While one exemplary embodiment of a crotch insert and absorbent composite is shown for purposes of illustration, a wide variety of portions and composites are optionally used within the scope of this invention.

Referring to the embodiment illustrated in FIG. 4, the cover 44a of the absorbent composite 44 is a liquid pervious layer which allows liquid to easily penetrate the cover to the layers beneath. Suitable materials for the liquid-permeable cover 44a include non-woven spunbond polypropylene of 15 gsm available from Avgol of Holon, Israel, wherein the non-woven material is zone coated, with its longitudinal central area being hydrophilic and its two longitudinal side areas being hydrophobic. The cover 44a may also be formed of an apertured three dimensional film or a combination of such film and a non-woven material. In accordance with an exemplary embodiment of the undergarment 20, the cover 44a is of the same size and shape as the barrier layer 44d and is disposed thereover. The cover 44a and the barrier layer 44d may be wider than the other layers such that the cover 44a and barrier layer 44d are directly connected to each other, thereby sandwiching the ADL 44b and absorbent core 44c therebetween. The cover 44a is glued to the barrier layer 44d with the ADL 44b, and the absorbent core 44c interposed therebetween.

In the exemplary embodiment shown in FIG. 4, an ADL 44b is used between the absorbent core 44c and the cover 44a. The ADL 44b is located over the thickened portion of the absorbent core 44, i.e., the portion where the fluid insult is expected to be initiated. The ADL 44b serves to manage, transport, accommodate and/or direct high volumes and flow rates of fluid into the core 44c.

The ADL 44b can be a thru-air bonded/carded web, a spunbond bi-component non-woven web, a web of crosslink cellulosic fibers, apertured 3D (three dimensional) film or the like. One particularly suitable material is a thru-air bonded bi-component fiber material with a finish surfactant available from PGI Non-Wovens of Landisville, N.J., which has an overall basis weight of 40 gsm. The bi-component fibers are made of a polypropylene inner core and a polyethylene outer sheath. The ADL 44b may be adhesively secured in place by any suitable construction adhesive, such as 34-373A adhesive available from National Starch and Chemical of Bridgewater, N.J.

Interposed between the ADL 44b and the barrier layer 44d is the absorbent core 44c. The absorbent core 44c, according to one embodiment, is made from untreated pulp and contains superabsorbent polymer. The absorbent composite 44, as shown in the embodiment illustrated in FIG. 1, lies transversely centered in the crotch insert 40, but is off-set toward the front portion in the longitudinal direction of the crotch insert, such that a portion of the absorbent composite 44 overlaps the belly area 25 of the front portion 20.

The absorbent core 44 is disposed above the barrier layer 44d. In the embodiment shown herein, the absorbent composite 44 is of a generally rectangular shape. If desired, it may have a central portion of increased thickness as compared to its side marginal portions. The thickened central portion of the absorbent core 44c may taper from the front to the rear. These structural features of the core 44c and its location ensures that the maximum thickness of the core is located at the anatomical position at which the urine insult from the wearer will be initiated.

The core 44c can be formed of any suitable material(s), such as an airlaid composite, containing pulp, superabsorbent particulates and/or fibers, and binders. The binders may be chemical or thermal.

In accordance with one exemplary embodiment, the core 44c is made up of mainly cellulosic fibers, e.g., wood pulp fluff made of up bleached sulfate wood pulp containing soft wood fibers, such as that available from International Paper of Tuxedo, N.Y., co-mingled with hydrogel polymer particulates (known as Super Absorbent Polymer or “SAP”) such as cross-linked polyacrylate IM-3900 available from BASF Corporation of Charlotte, N.C. If desired, these materials may be optionally wrapped in tissue. The amount of each absorbent material and SAP/fluff ratio depends on the size of the protective underwear, whether it is used for children (e.g., training pants) or for adults, and whether or not a transfer or fluid acquisition layer component is to be included in the absorbent composite 44. In this regard, the ADL 44b as shown herein may be optionally omitted from the undergarment 10.

In order to prevent the egress of fluid out through the non-woven sheets of the chassis, the crotch insert 40 also includes the heretofore identified barrier layer 44d. The barrier layer 44d is interposed between the core 44c and the chassis and is formed of a liquid impervious material, e.g., a microporous polyfilm which is vapor permeable (breathable). In the embodiment shown herein, the barrier layer 44d is of the same generally rectangular shape and size as the cover 44a and is located in the crotch insert 40 of the undergarment 10. In accordance with one exemplary embodiment, the barrier layer 44d is formed of a film of 0.5 mil polypropylene available from Pliant Corporation of Williamsburg, Va.

The crotch insert 40 of the absorbent garment also includes two sets of elastic threads 45 comprising at least one elastic strip each. The elastic threads 45 extend along the longitudinal direction of the crotch insert 40 for a distance along each side 43 of the crotch insert 40 such that each elastic thread 45 also overlaps the belly areas 25, 35 of the front 20 and back 30 portions. The elastic threads 45 are sandwiched between the cover 44a and barrier layer 44d and are attached using construction adhesive. In addition, the crotch insert 40 of the undergarment 10 includes an outer cover 44e, such as a non-woven layer, as illustrated.

It will be appreciated that the foregoing description of crotch insert 40 is provided for purposes of illustration of one exemplary embodiment. Alternative absorbent inserts are optionally utilized depending on performance, cost, comfort, and manufacturing considerations. For example, the crotch insert may be optionally provided with standing leg gathers to help promote fit and to prevent or reduce leakage.

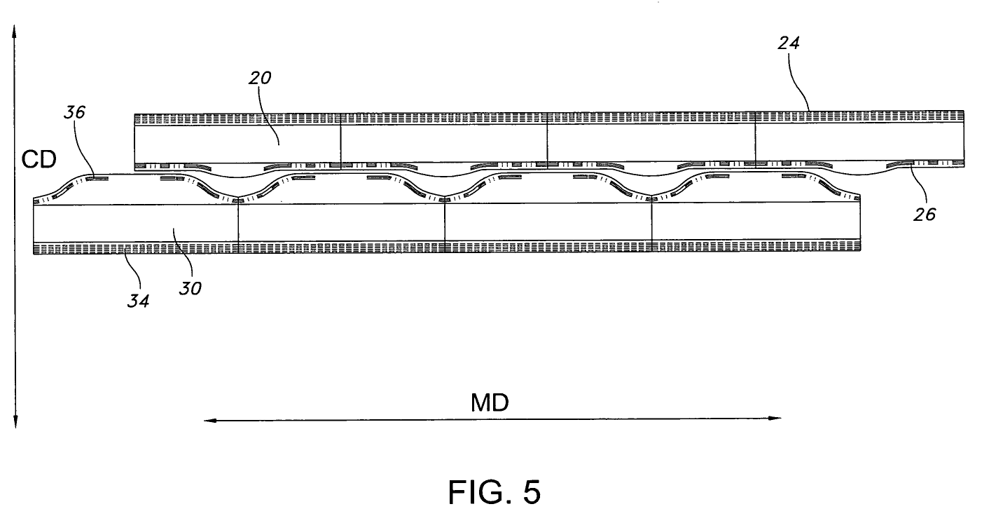

As shown in FIG. 5, the front portion 20 and back portion 30 may be formed from a single composite web of material that is wave-cut along the machine direction (MD) at the crotch edges 22, 32 of the front 20 and back 30 portions. The two portions are subsequently separated from one another in the cross-machine direction (CD), or longitudinal direction, to form a gap therebetween. The front 20 and back 30 portions are also indexed in the machine direction so that the highest amplitude of each wave-cut of the front 20 and back 30 portions are aligned. The crotch insert 40 is applied to these areas to form the absorbent undergarment 10, as shown in FIG. 1.

In another exemplary aspect of the invention, as shown in FIG. 1, the invention provides a protective undergarment 10 having a front portion 20 and a back portion 30, in which each portion 20, 30 has a stretchable waist area 11, 16, a stretchable leg area 13, 14 longitudinally spaced from the waist area 11, 16, and a stretchable belly area 12, 15 positioned between the waist area 11, 16 and the leg area 13, 14. The belly area 12, 15 is separated from the waist area 11, 16, thereby defining a substantially non-stretchable portion 50 therebetween. Further, the belly area 12, 15 is separated from the leg area 13, 14 thereby defining a substantially non-stretchable portion 51 therebetween.

By providing non-stretchable gaps or portions 50, 51, this aspect of the invention allows for each of the waist areas 11, 16, belly areas 12, 15 and leg areas 13, 14 to have different degrees of stretchability suited for fit around the waist, the belly, and the legs of the wearer, respectively. This allows the selection of elastics materials and/or the selection of their respective degrees of tension so that individualized fit characteristics can be provided at the waist, belly and legs. Thus, this configuration permits flexibility in the design and manufacture of the absorbent undergarment so as to provide improved performance and fit. The respective tensions applied at the waist, belly, and legs can be the same or differ. Also, the respective materials used for the waist, belly, and leg elastics can be the same or differ.

Additionally, by providing non-stretchable gaps or portions 50, 51 according to this exemplary aspect of the invention, the absorbent garment can be configured to approximate briefs and undergarments in terms of fit and comfort. For example, the gaps or portions 50, 51 allow the waist, belly, and leg elastic portions to be independently stretched by the wearer, for better comfort and fit. This exemplary feature also allows for independent stretching of one or more of the waist, belly, and leg portions as the absorbent undergarment is donned by the wearer. Further, less elastic material is required in the undergarment provided with gaps or portions 50, 51, thereby providing a cost savings.

The protective undergarment 10 can include non-stretchable portions 50, 51 that each have a width in the longitudinal direction of about 0.5 cm to about 3 cm, or preferably, the non-stretchable portions 50, 51 each have a width in the longitudinal direction of about 1 cm.

As shown in FIG. 1, the waist elastics 24, 34 and the leg elastics 26, 36 comprise elastic threads or filaments, whereas the belly elastics 25, 35 comprise elastomeric film. It is also contemplated, however, that elastic threads or filaments may be used for the belly elastics and that elastomeric film may be used for the waist and leg elastics.

In another embodiment, the invention provides a protective undergarment 10 comprising a front portion 20 and a back portion 30 longitudinally spaced from one another. Further, each portion has a waist area 11, 16, a leg area 13, 14 longitudinally spaced from the waist area 11, 16 and a belly area 12, 15 positioned between the waist areas 11, 16 and the leg areas 13, 14. The belly areas 12, 15 include an ultrasonically bonded, stretchable film. A crotch insert 40 having a front end 41 fixed to the front portion 20 and a back end 42 fixed to the back portion 30 bridges a gap between the front 20 and back 30 portions, such that portions of the leg area 13 of the front portion 20, the leg area 14 of the back portion 30, and the crotch insert 40 together define leg openings 18. In this example, the stretchable film may be bonded on one side to a material, such as a non-woven material, and the other side remains exposed. The ultrasonic bonding of the belly areas 12, 15 results in a composite having a more cotton-like, or underwear-like, look and feel.

This embodiment also has front 20 and back 30 portions that are made up of inner portion layers 27, 37 and an outer portion layers 28, 38, with a stretchable elastomeric film sandwiched therebetween. In this example, the undergarment includes ultrasonic bonds for securing both the inner and outer portion layers and the elastics. Optionally, the belly areas 12, 15 may be uni-directionally stretchable in a direction across a wearer's belly, and/or the undergarment may include a front 20 portion and a back portion 30 that each have a wave-shaped pattern along crotch ends 22, 32 of the respective portions. Alternatively, the undergarment, as shown in FIG. 6, may have a back portion 130 with a wave-shaped pattern along a crotch end portion 132 of the back portion 130 and the front portion 120 has a substantially straight crotch end portion along the front portion 120. The stretchable films of the belly areas 12, 112 and 15, 115 are preferably ultrasonically bonded under tension.

Accordingly, referring to FIG. 1, the above-described embodiment provides two sheets of material (preferably non-woven) as the inner and outer layers. Within these two layers are waist elastics 34 which are comprised of either elastic strands or elastomeric film, such as laminate film. Adjacent to the belly areas 12, 15 are the leg areas 13, 14 that are comprised of elastic threads or elastomeric film. The waist areas 11, 16 and leg areas 13, 14 may be bonded using ultrasonic bonding or adhesive. With increased retractile force in the waist and leg areas there is added tension that keeps the underwear in place and aids in fluid handling and fit. Thus, all the elastomeric characteristics of the product (elastic threads and/or elastomeric film) are optionally contained within the two sheets of material 27, 37 and 28, 38, which form composites. These composites are attached to the crotch insert 40 to create three larger subassemblies, one being the crotch insert 40, and the other two subassemblies making up the front 20 and back portions 30.

Optionally, in a variation of this embodiment, the front 20 and back 30 portions are made breathable in the waist, belly and/or leg areas of the undergarment. For example, the front 20 and back 30 portions may optionally be made breathable by utilizing suitable elastomeric film, such as laminate film. The front 20 and back 30 portions could also be rendered breathable by providing perforations or otherwise providing holes or openings or apertures so as to provide a breathable undergarment. Such breathability provides enhanced comfort for the wearer. Alternatively, during the lamination process, the front 20 and back 30 portions may be rendered breathable.

As discussed with respect to FIG. 1, embodiments of the invention can include an undergarment having waist elastics 24, 34, in which elasticized constructions for the waist area 11, 16, such as foam, lycras®, fluted elastic, and similar materials are used. It is recognized, however, that although these designs may be suitable for the purposes for which they are intended, they may not provide optimal fit and comfort for the consumer under certain circumstances and in some embodiments. Rather, it has been discovered that improved appearance, better performance and better fit can be attained by modifying the design as well as the materials of construction particularly for the waist area in selected embodiments.

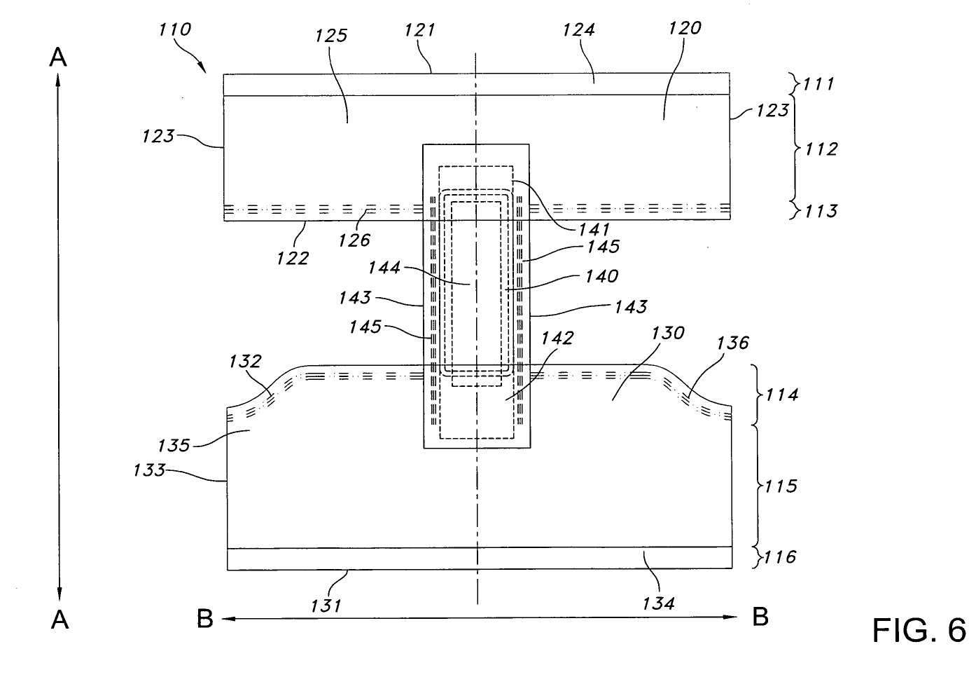

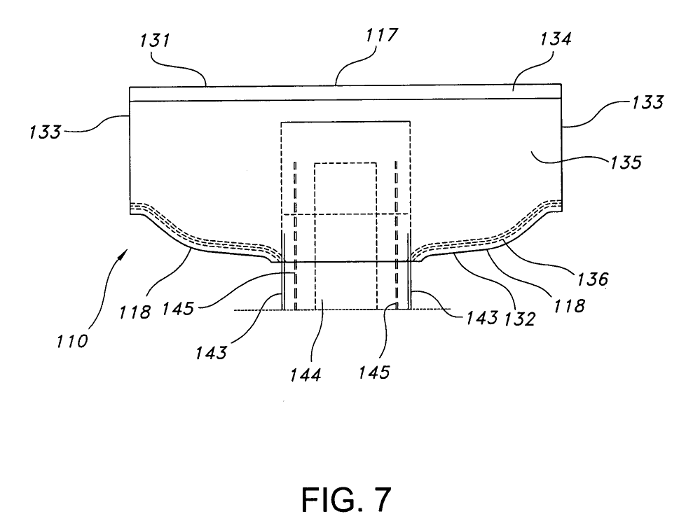

Thus, in another aspect, the invention provides for an embodiment of an absorbent protective undergarment 110, as shown in FIG. 6. In this embodiment, the front portion 120 may include a substantially straight waist end 121 along a transverse direction, a substantially straight crotch end 122 longitudinally spaced from the waist end 121 and running in a transverse direction, and two substantially straight sides 123 that extend between the waist end 121 and the crotch end 122. As shown in FIG. 6, the waist elastics 124, 134 are included as elastomeric film. FIG. 7 illustrates the fully assembled absorbent undergarment of FIG. 6, similar to the illustration of the embodiment of FIG. 1 shown in FIG. 2.

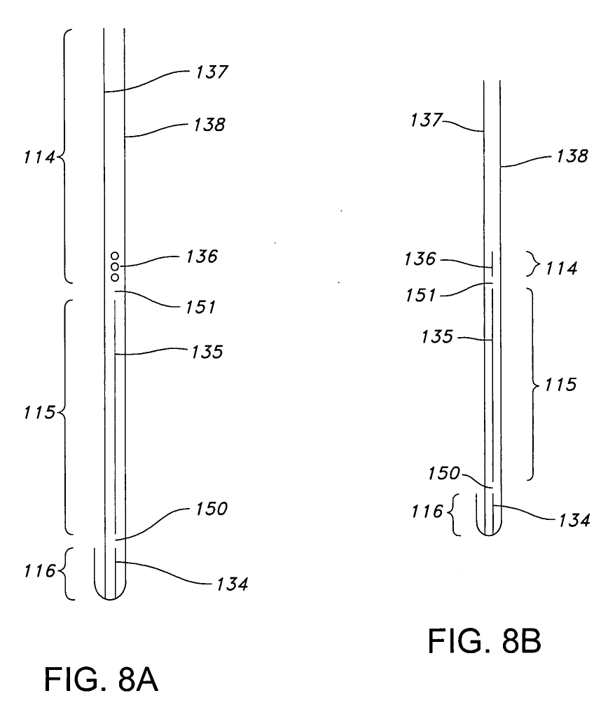

Referring now to FIGS. 8A and 8B, which are side cross-sectional views of the back portion 130 of the undergarment of FIG. 7, the undergarment 110 includes a back outer portion layer 138 and a back inner portion layer 137. Although not shown in FIGS. 8A and 8B, the front portion 120 is similarly designed, as reflected in FIG. 3. (For simplicity, the front portion is not illustrated, but has a side view that is essentially a mirror image of the back portion, as seen in FIG. 3). The back portion 130 comprises a back outer portion layer 138 and a back inner portion layer 137. Interposed between the back outer portion layer 138 and inner portion layer 137, the undergarment 110 includes a stretchable waist area 116, a stretchable belly area 115, and a stretchable leg area 114. The stretchable waist area 116 includes waist elastics 134 comprising elastomeric film. As illustrated in FIG. 8A, the back outer portion layer 138 is folded over the waist elastics 134 and the back inner portion layer 137. The stretchable belly area 115 includes elastomeric film for the belly elastics 135. As shown in FIG. 8A, stretchable leg area 114 can include elastic threads or filaments.

Alternatively, the absorbent protective undergarment, as shown in FIG. 8B, includes elastomeric film for each of the waist elastics 134, belly elastics 135, and leg elastics 136 interposed between outer portion layer 138 and inner portion layer 137 of the back portion 130. In each of the embodiments of FIGS. 8A and 8B, waist elastics 134 and the leg elastics 136 may be attached to the back portion outer and inner layers 138 and 137, respectively, by adhesive bonds or by ultrasonic bonds. As noted above, the front portion 120 is essentially a mirror image of the design of the back portion 130.

The waist, belly and leg elastics, for example as shown in FIGS. 3, 8A and 8B, correspond to the area of coverage and required tension for fit. There are number of combinations of elastic materials for the three corresponding areas within the inner and outer portion layers of the respective front and back portions. For example, the waist, belly and leg elastics could be provided in combinations of lycra/full flex and/or film, providing at least nine combinations. The outer portion layers of each of the front and back portions may also act as the carrier for the elastic materials. In addition, there may also be included a variety of combinations of bonding within the three corresponding front and back portion areas. For example, the waist, belly and/or leg areas may be bonded using a combination of ultrasonic bonding and/or adhesive for bonding each of the elastic materials to each of the respective areas.

In the embodiment shown in FIGS. 6, 7, 8A and 8B, the protective undergarment includes a waist opening 117, a front portion 120 and a back portion 130, each of which comprises a non-woven layer having a leg area 113, 114, a belly area 112, 115, and a waist area 111, 116. The non-woven layer extends proximal to the waist opening 117, and the stretch material is coupled to the non-woven layer and extends about the waist opening 117. The non-woven layer is folded over the stretch material at the waist opening 117 to capture the stretch material between portions of the non-woven layer.

Optionally, the undergarment stretch material includes a bi-directionally stretchable web having a width to thickness ratio of at least 3:1. The undergarment the stretch material can include elastomeric stretch film or at least one thread of an elastic filament. The undergarment stretch material may be attached to the non-woven by adhesive bonding, ultrasonic bonding, heat or embossing as well as by other attachment means.

In the embodiment as shown in FIGS. 6, 7, 8A and 8B, the protective undergarment comprises front portion 120 and back portion 130, each portion comprising an outer layer 128, 138 and an inner layer 127, 137 having different lengths. Each of the front 120 and back 130 portions has a waist area 111, 116 that includes a stretchable web having a width to thickness ratio of at least 3:1, a leg area 113, 114 longitudinally spaced from the waist area 111, 116, and a belly area 112, 115 positioned between the waist area 111, 116 and the leg area 113, 114.

This embodiment of the undergarment is designed in a configuration such that the longer of the inner layer 127, 137 and outer layer 128, 138 of the front 120 and back 130 portions is folded over the shorter of the inner 127, 137 or the outer 128, 138 layer of the front 120 and back 130 portions at the waist end to sandwich the stretchable web therebetween.

The undergarment of this embodiment includes elastomeric film as the stretchable waist material. Further, the undergarment stretchable web can be attached to the front and back portions by adhesive bonding, ultrasonic bonding, heat or embossing or other attachment means. Moreover, the stretchable web of the front 120 and back 130 portions may be either a single web of material divided into two pieces, one for each of the front 120 and back 130 portions, or it may be separate elastic webs, one set for each of the front 120 and back 130 portions. These embodiments incorporate elastomeric film as the waist elastics 134, such that the waist elastics 134 extend to cover the circumference of the product. In other words, when fully assembled, the undergarment 110 includes waist elastics 124 and 134 attached from end to end to form waist opening 117. Thus, the position and dimensions of the elastomeric film in relation to the rest of the finished product are advantageous.

These improved waist designs as described above and as shown in FIGS. 8A and 8B improve upon conventional waist designs that include materials, such as LYCRA®, that tend to fold down during usage due to the thickness of the LYCRA®, e.g. 420-900 dtex or foam-polyurethane, which has a thickness of 1 mm to 3 mm. Such materials tend to have a very low recovery force. This embodiment minimizes the waist area's tendency to roll in or out, which results in discomfort. With the combination of the waist elastics 124, 134 comprising stretch film and the waist area having the outer layer 128, 138 folded over the inner layer 127, 137, for example, as shown in FIGS. 8A and 8B, this embodiment achieves an even better performance and better fit, than either of these features alone.

In another aspect, the absorbent undergarment can be formed by the mating of a front portion 120, a back portion 130, and a crotch portion 140 such that the crotch portion 140 bridges a gap between the front 120 and back 130 portions. Specifically, the method includes forming from at least one continuous web of material into outer front and back portions each having a waist end and a crotch end longitudinally spaced from the waist end. A stretchable film, under tension, is applied onto each of the outer front and back portions at a belly area of each of the front and back portions. An ultrasonic bond is applied to the stretchable film and the outer front and back portions. Finally, a front end of a crotch insert is fixed to the front portion and a back end of the crotch insert is fixed to the back portion thereby bridging a gap between the front and back portions.

With reference to FIGS. 1, 2, 3 and 5, the method for forming the exemplary undergarment of FIG. 1, includes the following steps. First, a first substrate is provided in the machine direction (MD), shown in FIG. 5, to form an outer layer. A next step includes applying elastic film, provided in two pieces, under tension onto the outer layer to form the belly elastics for each of the front and back panels. Elastic threads (and/or elastomeric film) are next adhesively bonded, under tension, to the outer layer to form waist elastics and leg elastics. Then, a second substrate is provided in the machine direction (MD) to form an inner layer for mating with the outer layer, in which the inner layer has a width in the cross-machine direction (CD) less than the outer layer. Next, the leg elastics are deactivated, preferably by cutting or removing a portion of the leg elastics located where the overlap of the crotch portion will ultimately be placed. Then, the elastomeric films of the front and back portions are ultrasonically bonded to the inner and outer layers and the extra width of the outer layer is folded over and bonded to form outer front and back panels that overlap the inner front and back panels. The assembly is then cut along its length to longitudinally separate the front and back portions. The next step includes indexing, or offsetting, the front and back portions to align the front and back portions, from the position as shown in FIG. 2 to the positions shown in FIG. 1. Next, the absorbent crotch portion is applied to bridge the gap between the front and back portions, as shown in FIG. 1. The crotch portion is attached along the sides of the front and back portions nearest the belly bands. Finally, the front and back portions are connected along their sides.

The method may optionally include steps of forming from at least one continuous web of material inner front and back portions each having a waist end and a crotch end longitudinally spaced from the waist end for mating with the outer front and back portions, and mating the inner front and back portions to the outer front and back portions with the stretchable film sandwiched therebetween, prior to the step of applying the ultrasonic bond. A further optional step may include deactivating the leg elastics of the front and back portions, prior to fixing the crotch insert to the portions, by cutting or alternatively removing a portion of the leg elastics at locations where the crotch insert overlaps the front and back portions.

In still another aspect, the invention provides a method of manufacturing a protective undergarment including the step of forming from at least one continuous web of material, such as non-woven material, front and back outer portions. The process is directed to assembling a stretch material located at the waist area of the finished product. The stretch material is preferably stretchable in the cross-machine direction (CD) and in the machine direction (MD) and, unlike conventional methods, does not require turning of the elastic material or use of “cut and place” equipment.

In an embodiment according to this aspect, the invention includes assembling two web materials, such as a non-woven backsheet, or other outer cover material, and a CD/MD stretch material with retractive force qualities. Next, the method includes elongating the stretch material along its length, or machine direction, and then attaching the material to the area of the non-woven which will become the waistband of the product. In the process, the front waistband is constructed on the drive or operator side of the machine and the rear waistband is constructed on the opposite side of the machine. A next step includes folding the non-woven over to completely cover the stretch material or, alternatively, including an additional non-woven component to be placed over the stretch material. Alternatively, one side of the stretch material may be uncovered if the elastic material has sufficient aesthetic and tactile qualities to make it comfortable against the wearer of the product.

Optionally, the elastic material is attached to the other web at intermittent points, so that less than 100% of the area of the elastic web is attached to the non-woven. In addition, there may be regular pattern with distinct areas of no attachment followed by areas of intermittent attachment.

In further steps, the method includes coupling a first continuous web of stretch material to the front outer portion along a machine drive side of the non-woven material to form a front waistband. The web of stretch material may be, for example, a bi-directionally stretchable web having a width to thickness ratio of at least 3:1 (as compared to an elastic thread having a thickness ratio of about 1:1), or other stretchable film. Alternatively, the stretch material may, for example, include at least one elastic thread such as LYCRA® spandex.

In another step, the method includes coupling a second continuous web of stretch material to the back outer portion along a machine operator side of the non-woven material to form a rear waistband. The coupling of the stretch material to the non-woven may be performed by application of adhesive bonds, ultrasonic bonds, heat or embossing or other attachment means.

In an optional step, the method may include the step of elongating the stretch material along its length prior to coupling the material to the front and back outer portions. In yet another optional step, the method may include folding a portion of the non-woven material over the front and rear waistbands and connecting the front and rear waist bands to form a continuous waist opening.

Though the manufacturing steps described and claimed herein are optionally performed in the order described or claimed, they can be performed in any order, depending upon the equipment and processes selected and the preferences of the manufacturer.

In an alternative embodiment according to this aspect, the invention provides a method of manufacturing a protective undergarment including the step of forming from at least one continuous web of non-woven material front and back outer portions. The method further includes the steps of coupling a first continuous web of stretch material having a width to thickness ratio of at least 3:1 to the front outer portion along a machine drive side of the non-woven material to form a front waistband and coupling a second continuous web of stretch material having a width to thickness ratio of at least 3:1 to the back outer portion along a machine operator side of the non-woven material to form a rear waistband. The webs of stretch material may be coupled to the non-woven by applying adhesive bonds, ultrasonic bonds, heat or embossing, for example. Further, the method includes the step of connecting the front and rear waist bands to form a continuous waist opening.

In an optional step, the method of further comprises elongating the stretchable web along its length prior to coupling the web to the front and back outer portions. In additional optional steps, the method may include the steps of folding each of the front outer and back outer portions over the stretchable webs to cover the web, adding an additional non-woven web of material over the stretchable web to cover the web, and/or forming from a second continuous web of non-woven material front and back inner portions and sandwiching the first stretchable web between the inner front portion and the outer front portion and the second stretchable web between the inner back portion and the outer back portion.

The method may also include the optional steps of attaching the stretchable web to the front and back outer panels at intermittent points of attachment so that less than 100% of the stretchable web is attached to the front and back portions and/or creating repeating patterns from the points of attachment.

Still yet other optional steps of the method include forming the front and back portions with a repeating wave-shaped pattern along a crotch end of the portions and/or indexing the front and back outer portions to align a peak of a wave of the wave-shaped pattern with a peak of an opposing wave of the wave-shaped pattern, for example, as shown in FIGS. 1 and 6.

Alternatively, referring to the embodiment shown in FIG. 7, each leg opening 118 may have a curved edge at the crotch end 122 of the front portion 120; a curved edge at the crotch end 132 of the back portion 130; and an intermediate, substantially straight edge along the sides 143 of the crotch insert 140. The undergarment chassis is thus configured such that when it is worn it covers a portion of the wearers legs beneath the buttocks, while a front portion of the wearer's leg where the leg meets the torso is exposed. The undergarment therefore allows increased exposure of the front portion of the wearer's legs without sacrificing coverage of the wearer's buttocks.

In yet another aspect, and according to yet another embodiment, the invention is directed to a protective undergarment 210 comprising a front portion and a back portion each having a waist area, a leg area longitudinally spaced from the waist area, and a belly area between the waist area and the leg area. The undergarment further includes a crotch insert having a front end overlapping with the front portion and a back end overlapping with the back portion at the belly area.

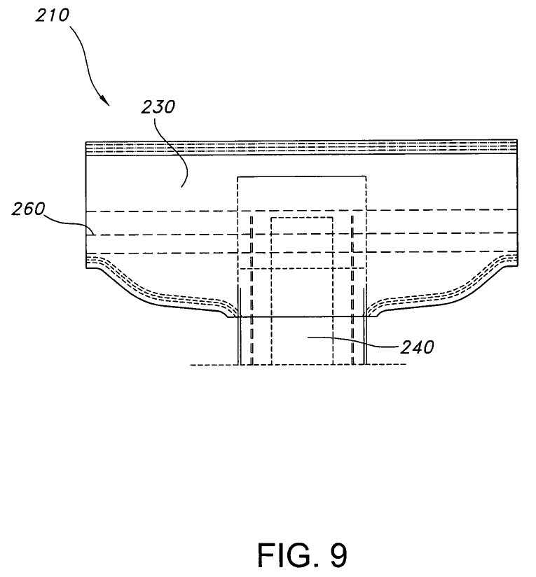

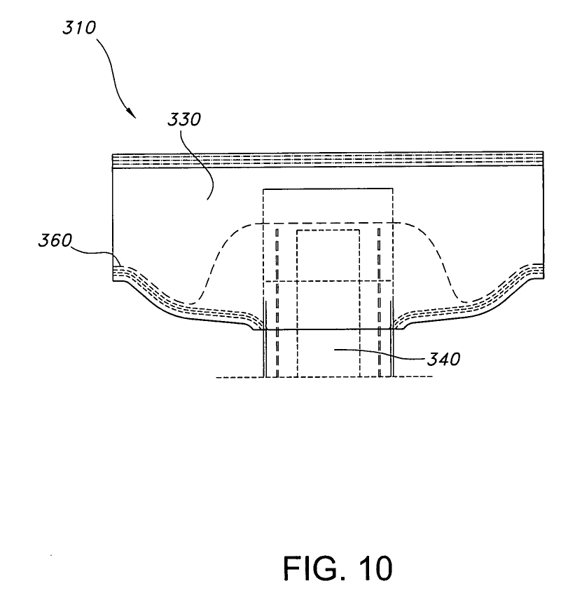

Referring specifically to FIGS. 9 and 10, the undergarments 210, 310 include a front end of the crotch insert fixed to the front portion (not shown) and ultrasonic bonds 260, 360, which can be applied in either the machine direction or the cross-machine direction, fixing the back end of the crotch insert 240, 340 to the back 230, 330. The portions of the leg area of the front portion, the leg area of the back portion, and the crotch insert together define leg openings.

Optionally, the ultrasonic bonds 260 comprise at least one line substantially parallel to a waist end of the back portion, as shown in FIG. 9, which illustrates three parallel lines. Preferably, the ultrasonic bonds of this embodiment comprise from one to three lines that are substantially parallel to the waist end of the back portion. Alternatively, the ultrasonic bonds 360 may comprise at least one repeating pattern that is at least partially aligned with the profile of a crotch end of the back portion of undergarment 310, as shown in FIG. 10.

According to this embodiment, the ultrasonic bonds are optionally applied to an area covering less than about 95% of the stretchable material. Optionally, the ultrasonic bonds are applied to an area covering less than about 25% of the stretchable material. Further, the undergarment can optionally include belly areas of the front and back portions having stretchable material.

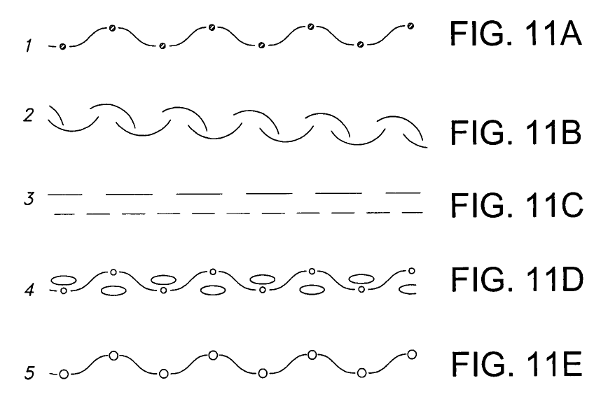

Exemplary patterns for the ultrasonic bond are illustrated in FIGS. 11A through 11E. As shown, the ultrasonic bonds may comprise a pattern of non-contiguous repeating elements. It is believed that by using non-contiguous patterns, the undergarment may be made more conformable. That is, the areas of non-contiguous bonding may be more easily bendable than in areas that are contiguously bonded and that are bent through the bonded area. However, contiguous patterns may also be used.

As shown in FIGS. 11A through 11E, the bond patterns may include combinations of lines, arcs, circles, ovals and dots, for example, among the elements that make up the pattern or patterns. The patterns are optionally repeated.

In a related aspect, the invention provides a method of manufacturing a protective undergarment. The method includes the steps of forming front and back undergarment portions, each having a belly area comprising a stretchable film and spacing the front and back undergarment portions longitudinally, or in the CD direction, from one another. The method further includes the steps of fixing a front end of a crotch insert to the front portion and fixing a back end of the crotch insert to the belly area of the back portion by ultrasonic bonds, to bridge a gap between the front and back undergarment portions.

Optionally, the method can include the step of fixing a back end of the crotch insert to the belly area of the back portion by applying at least one repeating ultrasonic bond pattern that is at least partially aligned with the profile of a crotch end of the back portion. Alternatively, the crotch insert can be affixed to the belly area of the back portion by applying an ultrasonic bond that includes at least one line parallel or substantially parallel to a waist end of the back portion. Preferably, the ultrasonic bond includes from one to three lines that are parallel to the waist end of the back portion. A further optional step includes fixing a back end of the crotch insert to the back portion by applying an ultrasonic bond to an area covering less than about 25% of the stretchable film. The ultrasonic bond can also, more preferably, be applied to an area covering less than about 95% of the stretchable film. The ultrasonic bond can be applied in either the machine direction or in the cross-machine direction. Further, in process, the application of the ultrasonic bond can be made either before or after the wave cut is applied to the front and back portions.

Accordingly, the above-described bonding process includes the sealing of two portions of the product, the belly elastic, which can be stretched and the crotch insert, which also may be stretched. The belly elastic materials that can be bonded together are preferably elastomeric stretch film, e.g. formed in-line, or a pre-laminated film, e.g. formed off-line. Further, the method is preferably used for protective underwear undergarments, but it could also be utilized for producing training pants, diapers or feminine hygiene products or other absorbent products.

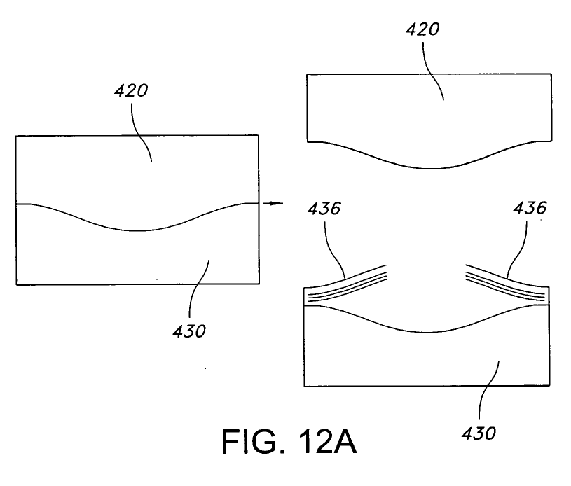

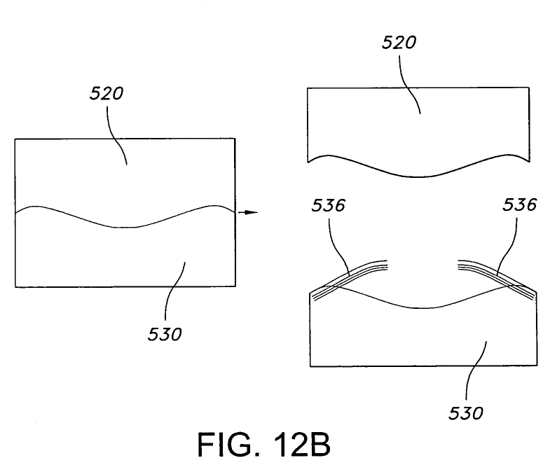

As described in greater detail below with reference to FIGS. 12A and 12B, the wave cut pattern will optionally cover about 0.5 to 1.5 of the period of the cycle in the transverse direction. In other words, the width of the front and back portions measured in the transverse direction will correspond to about 0.5 to about 1.5 of the cycles of the wave cut.

More specifically, and as shown in FIGS. 12A and 12B, the undergarment optionally has differing degrees applied to the wave-cut for forming the front 420, 520 and back 430, 530 portions of the illustrated embodiments. The left view shows the sheet from which the front 420, 520 and back portions 430, 530 are formed, and the right view illustrates the front 420, 520 and back 430, 530 portions after the portions have been cut and separated in the machine direction. FIG. 12A may be referred to as a 0.5 sinusoidal wave-cut because the width in the transverse direction corresponds to about 0.5 cycles. Also, FIG. 12B may be referred to as a 1.5 sinusoidal wave-cut because the width in the transverse direction corresponds to about 1.5 cycles. FIGS. 12A and 12B also illustrate another feature of the present invention, that is the deactivation of the leg elastics 436, 536 of the back portion. In FIGS. 12A and 12B, only the leg elastics 436, 536 of the back portion 430, 530 are shown, however, deactivation may also be applied to the leg elastics of the front portion, for example as shown in FIG. 1. Deactivation occurs at the location where the crotch insert (not shown) will ultimately overlap the front 420, 520 and back portions 430, 530. In other words, the elastics 36 may lose substantial stretchability in the transverse, or machine, direction when overlapped by the crotch insert, which is not substantially stretchable in the transverse direction. Alternatively, and as shown in FIGS. 12A and 12B, the leg elastics 436, 536 may be deactivated by removal of a portion of the elastics at the location where the crotch insert is attached to the back portion 430, 530.

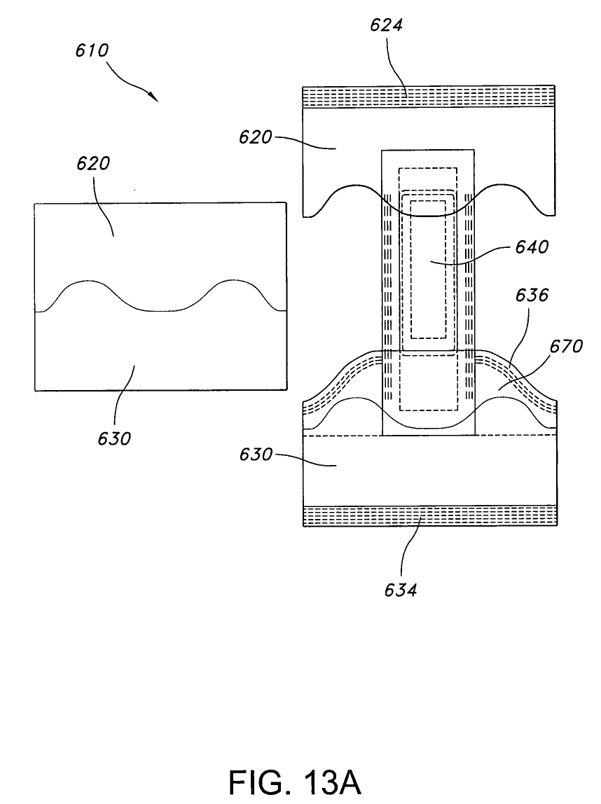

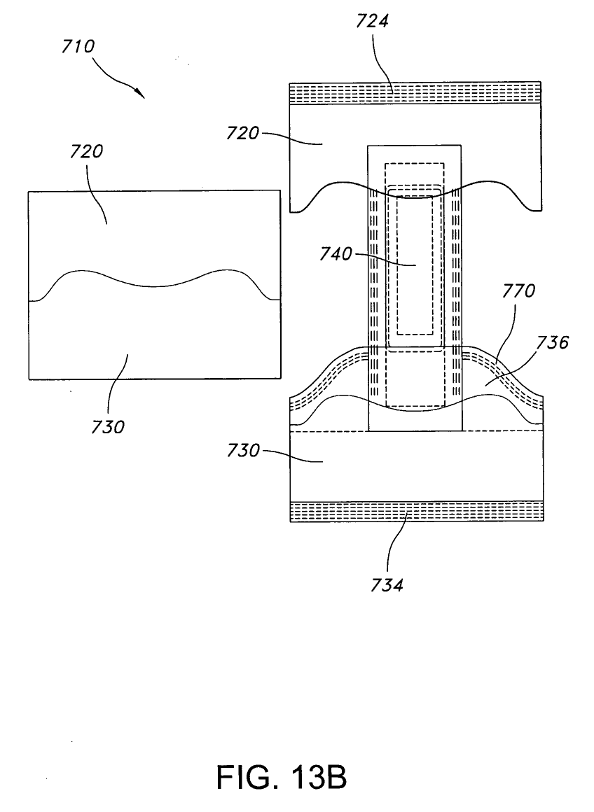

In still yet additional alternative embodiments, FIGS. 13A and 13B also illustrate exemplary repeating wave-cut patterns of the front 620, 720 and back portions 630, 730. In addition, FIGS. 13A and 13B further include the additional feature of leg portion 670 and 770, which are made of non-woven material and include leg elastics 636, 736. As shown, front portion 620, 720 is separated from back portion 630, 730, such that the crotch insert 640, 740 bridges a gap and joins together the front 620, 720 and back 630, 730 portions. The back portion 630, 730 further includes leg portion 670, 770 attached thereto, with the crotch insert 640, 740 attached at both the leg portion 670, 770 and the back portion 630, 730. This configuration provides the leg portion 670, 770 to be interposed between the crotch insert 640, 740 on the inside and the back portion 630, 730 on the outside. The leg portion 670, 770 extends longitudinally from the back end of the crotch insert 640, 740 towards the front portion 620, 720, as shown in FIGS. 13A and 13B.

With this configuration, less elastic material is required for the back portion 630, 730, thereby resulting in cost savings. Further, the configuration provides additional coverage for the front portion 620, 720 along the side seams of the front portion, as shown in FIGS. 13A and 13B.

The embodiment as described above is designed to minimize and/or eliminate waste of valuable stretch material, including stretch film, film-laminated non-woven and/or tri-laminated non-woven to replace non-woven laminated elastic threads. The design cutout conforms to the wearer's body providing an overall better fit and improved fluid handling. As noted previously, the attachment of the back panel to the crotch insert creates a dead-ended zone along the attachment. Due to this zone the front and back portions are designed to have stretch material placement that provides strategic stretch points around the waist and sides of the buttocks while conserving this material. As shown, the front portion 620, 720 is cut to resemble real underwear and to provide coverage over the crotch insert with better fitting contours around the thighs of the wearer. Leg portions 670, 770 provide increased coverage to the back portion 630, 730 while the front portion 620, 720 can be provided with less material. Optionally, the length of the side seams can be the same so as to be aligned during bonding by ultrasonic bonding, glue, or heat seal, for example. Further, the cut could be as a 0.5 (man favoring design) or 1.5 (female favoring design) sinusoidal wave as described above in connection with FIGS. 12A and 12B. In either case, the amplitude of the cycle should be the greatest at the point where the front panel is attached to the core insert.

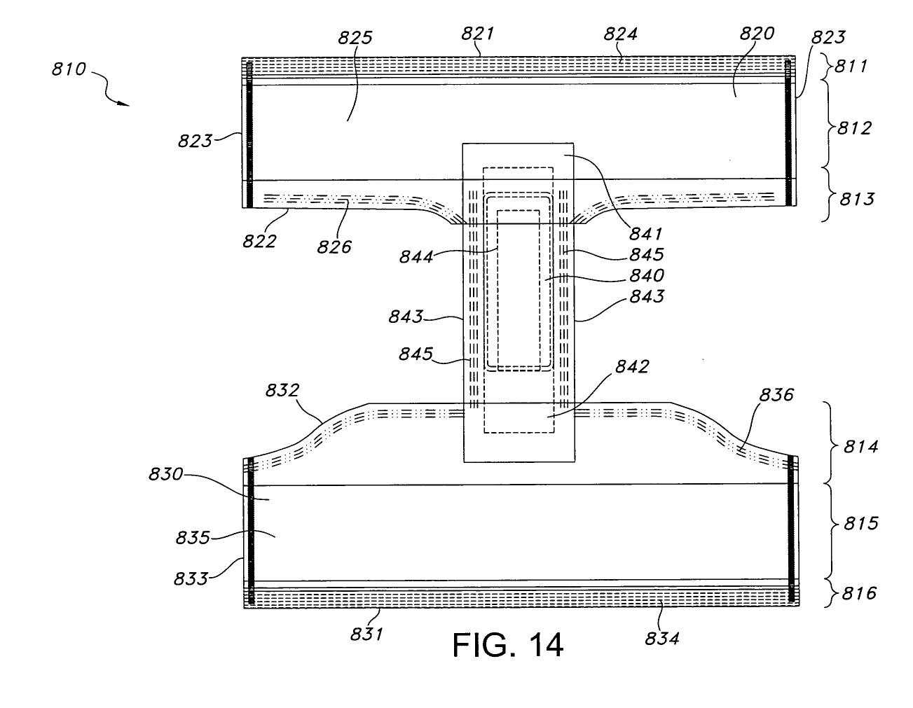

In still yet another aspect, according to an exemplary embodiment of the invention, the invention is directed to a method of manufacturing a protective undergarment. In this embodiment, the manufacturing method may include several steps performed in a substantially continuous operation. A first step includes forming front and back portions of the undergarment from at least one continuous web of material. Each of the front and back portions have a waist end and a crotch end that are longitudinally spaced apart and have substantially straight edges. In another step, the front end of the crotch insert is fixed to the front portion while the back end of the crotch insert is fixed to the back portion. The crotch insert thus bridges the gap between the front and back portions. In yet another step, material from the front and back portions is removed at the crotch ends of at least one of the portions to form wave-shaped edges, for example, as shown by crotch ends 822 and 832 in FIG. 14. Though the manufacturing steps described herein are preferably performed in the order described, they can be performed in any order, depending upon the equipment and processes selected and the preferences of the manufacturer.

In an embodiment according to this aspect, substrates are provided that are used to form the front and back inner and outer portion layers. The continuous web that makes up the front and back inner portions is loaded into a processing machine. As the inner layer material moves through the machine, the web is cut into two web portions along a straight line longitudinally extending in the machine direction (direction B-B shown in the figures), the two webs comprising the web portions that form each of the front inner portion and the back inner portion. Each of these front and back inner portions have a waist end and a crotch end longitudinally spaced from the waist end and have a substantially straight edge. Similarly, the outer portion layers are also loaded into the machine and likewise cut into two webs. The webs thus formed are generally rectangular in shape. Optionally, the back portion outer layer is longer in the cross-machine direction (direction A-A shown in the figures) than the front portion outer layer.

The front and back inner web portions are processed through a series of rollers after which waist elastics, belly elastics and leg elastics are applied. The elastics, which may optionally be applied in a pretensioned state, are then coated with adhesive, such as glue applied by strand coating glue heads. The leg elastics are applied so as to align with the contours of the intended finished undergarment's crotch edges. In an alternative embodiment, one or all of the waist elastics, belly elastics and leg elastics may comprise elastomeric material which is applied by ultrasonic bonding of the elastomeric material to the outer front and back portions instead of adhesive.

Then, the inner front portion web is “married-up” with the outer front portion web portion as the inner and outer portion webs are combined by a combining drum, thus sandwiching the elastic threads therebetween. The term “married up” is used herein to mean that the inner and outer web portions are substantially aligned and at least partially joined together to form a single portion or panel. After being combined by the combining drum, the front portion passes through a roller and under the insert attachment glue head at which point adhesive is applied to the front portion by a speed coat slot nozzle glue head. The adhesive is applied in a rectangular region so as to correspond to the overlap between the crotch insert and the front portion, as discussed in more detail below.

After the front and back portion inner and outer portion webs are married up, a thin strip of the crotch end of the back portion is removed to deactivate a section of the leg elastics of the front and/or back portion. The strip that is removed includes some of the web material and may also include portions of the leg elastics disposed in the area where the crotch insert is to be attached on the back portion of the undergarment. In other words, the leg elastics, which may include threads or filaments, are optionally applied to the front and/or back portions along a continuous length. The removal of a portion of the crotch end of the front and back portions may therefore remove a portion of the leg elastics in the location at which the crotch section is fixed to the front and back portions of the undergarment.

The step of removing a portion of the crotch end of the front and back portions therefore optionally involves retaining the crotch end of the front and back portions in a crotch insert fixation region. This is optionally accomplished by removing portions of the crotch end of the front and/or back portions on opposite sides of the crotch insert fixation region. Though this step can be performed in any order, in some embodiments the step of removing a portion of the crotch end of the front and/or back portions is advantageously performed before the step of fixing the front end of the crotch insert to the front portion and the back end of the crotch insert to the back portion.

Adhesive is next applied by a speed coat slot nozzle glue head. The adhesive is applied such that the entire area that the crotch insert will cover on the front and back portions is coated. While the adhesive is applied so as to ensure that as much of the peripheral edges of the crotch insert are adhered to the front and back portions, the adhesive is also applied so as to minimize adhesive leakage beyond the overlapping regions of the crotch insert with the front and back portions, respectively, from overspray or over application of the adhesive. Any such leakage may result in unintended adhesion of portions of the absorbent undergarment during the manufacturing and packaging processes.

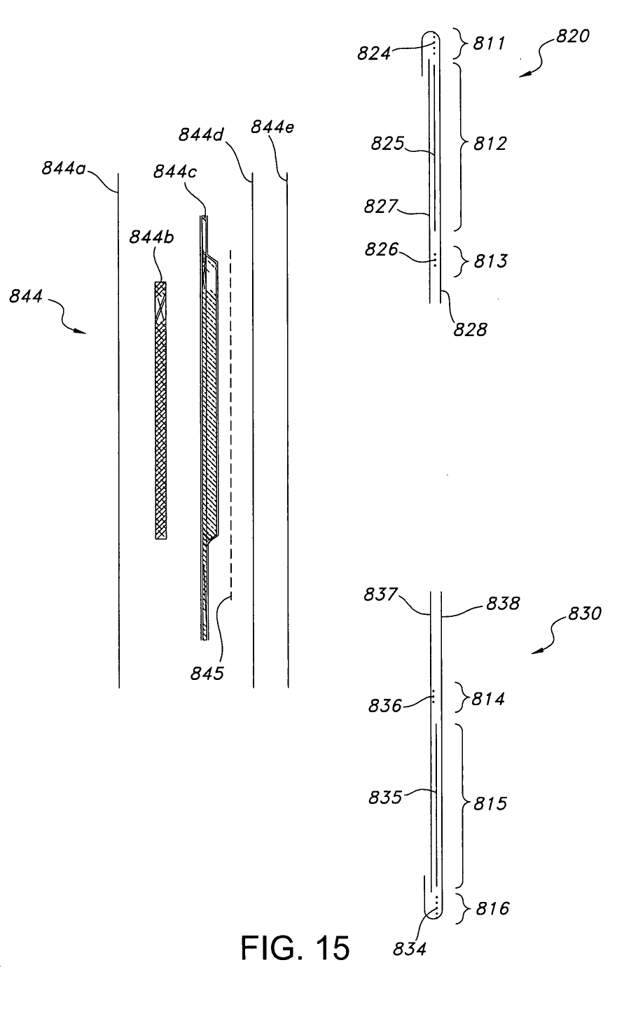

After the adhesive is applied, the front and back ends of the crotch insert are placed on the corresponding front and back portions of the undergarment. Upon drying, the adhesive holds the crotch insert firmly in place along the entire periphery where it overlaps the front and back portions of the absorbent undergarment. As shown, for example in FIG. 15, crotch insert 840, including crotch insert absorbent composite 844, is positioned on the inner, or body-side, of front portion 820 and back portion 830. Although not shown in FIG. 15, it is also contemplated that the crotch insert 840 may also be positioned on the outer, or garment-side, of each of front portion 820 and back portion 830. In either case, the crotch insert is fixed to the same body-side or garment-side of each of the front 820 and back 830 portions.

In alternative embodiments, after the crotch insert is applied, thin strips are removed from the crotch end of the front portion and the back portions. The strips that are removed include portions of the front and back portions, such that removal of these strips form the curved crotch edges of the front portion and back portions. This is exemplified in FIG. 14, which illustrates curved crotch edge 822 of the front portion 820 and curved crotch edge 832 of back portion 830 of the undergarment 810.

As shown in FIG. 14, resulting product 810 includes in the region of crotch edge 822 where the crotch insert 840 overlaps front portion 820, a substantially straight edge. This is in contrast to the undergarment 10, shown in FIG. 1, which illustrates undergarment 10 as including a substantially rounded region of curved crotch edge 22. Similarly, undergarment 810 also includes crotch edge 832 that includes a substantially straight edge from a center portion extending toward the sides 833, as compared, for example, to the crotch edge 32 of undergarment 10 of FIG. 1.

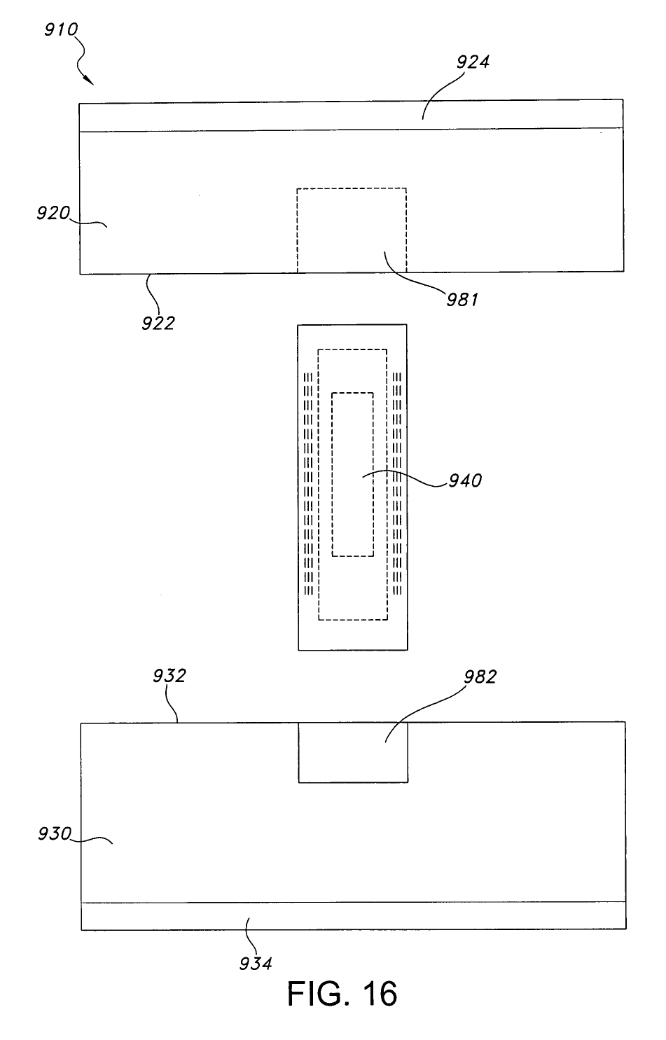

In an alternative embodiment, such as shown by the undergarment 110 of FIG. 6, the thin strip is removed only from the crotch end of the back portion. Another alternative embodiment, as shown in FIG. 16, includes undergarment 910 as having substantially straight edges for each of crotch edges 922, 932, respectively, of front portion 920 and back portion 930.

In an aspect of the invention, the crotch insert of the absorbent undergarment is then folded so as to align the waist end and opposing sides of the front portion with the waist end and opposing sides of the back portion. By connecting the front and the back portions along their sides, the arrangement forms a pants-shaped chassis having a waist opening and two leg openings. Individual products are then cut from the continuous web at the side connections of the undergarments.

Alternatively, the method may not include a folding step. In this case, the undergarment is not folded and the sides of the undergarment (and consequently the front and back portions) are not connected together during the manufacture of the undergarment. In such an embodiment, the undergarment optionally includes fasteners, such as adhesive tapes, hook and loop fasteners, or other conventional fasteners that are used in conventional disposable diapers that can be secured together at the time of use to form a pants-shaped chassis.

In another embodiment in which the outer web portions of the front and back portions are longer than the inner web portions, the longer outer webs are optionally folded over the shorter inner web, to provide an undergarment having the configuration as shown in FIGS. 3, 8A and 8B.

In another aspect, for example as shown in FIG. 16, the invention provides a protective undergarment 910 comprising a front portion 920 and a back portion 930 longitudinally spaced from one another, each of the front 920 and back 930 portions having a body-side and a garment-side. For illustration purposes, the body-side of undergarment 910 shown in FIG. 16 faces the viewer. The undergarment 910 further includes a crotch insert 940 having a front end 920 and a back end 930 and a body-side and a garment-side. In at least one embodiment, the body-side of the crotch insert front end is fixed to the garment-side of the front portion 920 and the garment-side of the crotch insert back end is fixed to the body-side of the back portion 930. This is exemplified by the schematic of FIG. 16, in which the area 981 defined by the dotted lines indicates the location of the crotch insert front end on the garment-side of front portion 920. Likewise, the area 982 defined by solid lines indicates the location of the crotch insert back end on the body-side of back portion 930. The crotch insert 940 bridges a gap between the front 920 and back 930 portions, such that the front portion 920, the back portion 930, and the crotch insert 940 together define leg openings when sides of the front portion 920 and back portion 930 are fixed together to form an assembled undergarment.

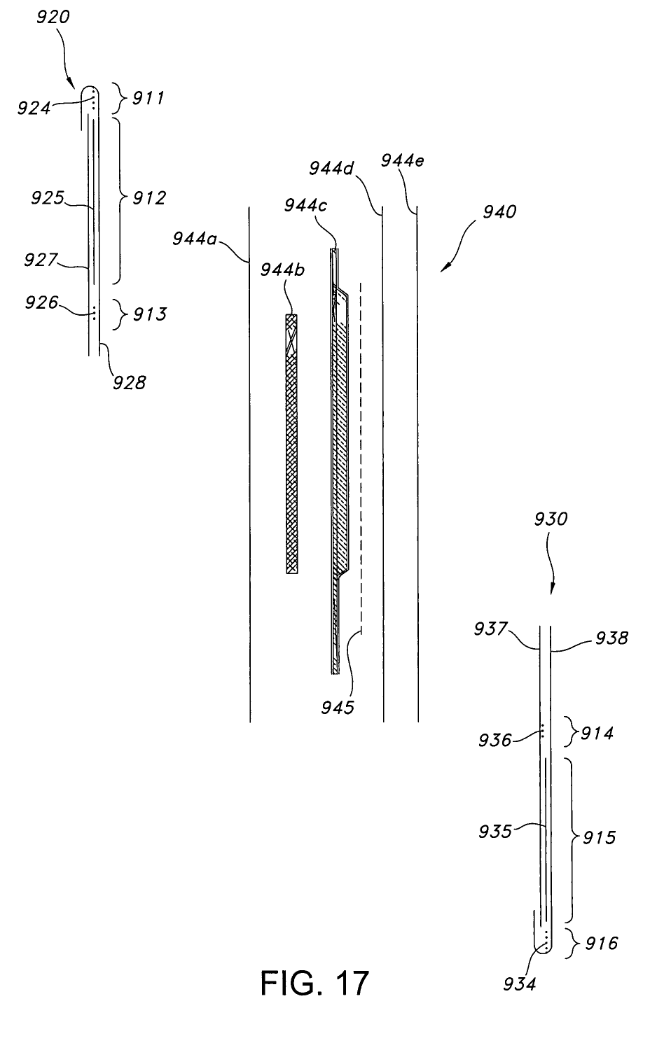

As shown in FIG. 17, the front 920 and back 930 portions may each comprise an inner portion layer 927 on the body-side with stretch material 924, 925 and 926 positioned on the inner portion layer 927. Optionally, and shown in FIG. 15, an outer portion layer 928 may also be provided on the garment-side of the front portion 920 with the stretch material 924, 925 and 926 positioned therebetween. The stretch material can include any of the stretch materials previously discussed herein. Inner and outer portion layers may include such materials as nonwoven materials.

Optionally, in an embodiment as shown in FIG. 16, the stretch material may be deactivated in the area 981 where the crotch insert is fixed to the front portion 920. Deactivation can be, for example, provided by a mechanical cut in the stretch material or by providing an area substantially devoid of stretch material. Such deactivation allows liquid from an insult to pass through the front portion 920, which is permeable, to the crotch insert 940 where it can be absorbed.

In another embodiment, in which the stretch material comprises an elastomeric film, such as laminate film, trilaminate film and the like, that is ultrasonically bonded to at least one of the inner and/or outer layers, the stretch material can also be deactivated by providing an area, such as area 981 of FIG. 16, that is substantially free of ultrasonic bonding of the stretch material to the inner and/or outer portion layers of front portion 920.

Optionally, the undergarment of this aspect may further include graphic design or designs incorporated on the front end of the crotch insert. For example, the graphic design can include a wide variety of shapes and characters aesthetically pleasing to wearers, such as cartoon characters for children. It has been found that the crotch insert cover, such as that shown in FIG. 17 as 944e which is made from a printed nonwoven or a polyethylene covered by a nowoven material to provide a better surface on which to print graphic designs than the crotch insert cover material and/or stretch material of the front portion.

Therefore, one of the advantages provided by the configuration of this aspect is that the design allows for easier incorporation of graphic designs on the front part of the product than if the crotch insert were applied to the body-side of the front portion. Further, because the outer non-woven is optional, a reduction in material cost is achieved.

In a further aspect of the invention, the invention provides a method of manufacturing a protective undergarment, including forming front and back undergarment portions that each have a body-side surface and a garment-side surface and spacing the front and back undergarment portions longitudinally from one another. Further steps include fixing a front end of a body-side of a crotch insert to the garment-side of the front portion and fixing a back end of a garment-side of the crotch insert to the body-side of the back portion. For example, such bonding in the back of the product may occur by fixing the crotch insert to either the body-side of the stretch film or the inner portion layer. Likewise, such bonding in the front of the product may occur by fixing the crotch insert to either the garment-side of the stretch film or, if the optional outer portion layer is utilized, to the outer portion layer. By the front and back end portions of the crotch insert to the front and back portions as described, the crotch insert bridges the gap between the front and back undergarment portions. Fixing the front end of a body-side of the crotch insert to the garment-side of the front portion can comprise, for example, adhesively bonding the front end of the crotch insert to the garment-side of the front portion or ultrasonically bonding the front end of the crotch insert to the garment-side of the front portion.

The method may optionally further include deactivating a section of stretch material included in the front portion where the crotch insert is fixed to the front portion, such as by mechanically cutting the stretch material or providing an area devoid of stretch material.

Other optional steps include ultrasonically bonding the front portion during the forming step. In such a case, deactivating the stretch material, for example elastomeric film, in the deactivation area includes zoning a section of stretch material so that is free of ultrasonic bonding where the crotch insert is fixed to the front portion.

In still yet another aspect, the invention for example as shown in FIGS. 16 and 17, provides a protective undergarment 910 comprising a front portion 920 having a body-side and a garment-side, the front portion 920 comprising a stretch material included thereon and a crotch insert 940 having a body-side and a garment-side. The body-side of the crotch insert 940 has a first end fixed to the garment-side of the front portion 920 and the stretch material is deactivated in an area, such as area 981, overlaps and is where the crotch insert 940 fixed to the front portion 920. The undergarment 910 further may include a back portion 930 having a body-side and a garment-side, wherein the garment-side of the crotch insert 940 has a second end fixed to the body-side of the back portion 930. Alternatively, the back portion 930 may be fixed to the garment-side of the back portion 930.

As discussed above, deactivation of the stretching ability of the absorbent undergarments according to the various embodiments of the invention may be achieved by mechanically cutting the stretch material or by providing an area substantially devoid of stretch material. Alternatively, in embodiments in which the undergarment includes stretch material, such as elastomeric film, ultrasonically bonded to at least one of the inner and/or outer portion layers, the film may also be deactivated by including an area substantially free of ultrasonic bonding to the inner and/or outer layers.

While the invention includes the various embodiments described above, other embodiments of the invention may also include features as shown and described in U.S. patent application Ser. No. 10/617,218 to Glaug et al., filed on Jul. 10, 2003 and Ser. No. 11/236,994 to Babusik et al., filed on Sep. 28, 2005, which application is incorporated herein by reference. Each of the embodiments of the present invention is expected to provide equal or better functional performance and customer acceptance at a lower cost as compared to conventional protective undergarments. In particular, equal or improved performance is expected with respect to characteristics such as leakage control, coverage in the front, coverage in the back, fit at the waist, fit at the legs, overall fit, comfort, and ease of donning and doffing the undergarment.

While preferred embodiments of the invention have been shown and described herein, it will be understood that such embodiments are provided by way of example only. Numerous variations, changes and substitutions, including but not limited to the materials that make up the various components of the absorbent protective undergarment, will occur to those skilled in the art without departing from the spirit of the invention. Accordingly, it is intended that the appended claims cover all such variations as fall within the spirit and scope of the invention. For example, although the invention has been described as an absorbent undergarment having a three-component configuration, i.e. a front portion, a back portion and a crotch insert, the absorbent undergarment may have a one-piece rectangular or hour-glass configuration. Also, the steps of the fabrication methods described herein can be performed in various orders, depending on manufacturing, cost, and design considerations.

What is claimed:

1. A protective undergarment comprising:
a waist opening;
a front portion and a back portion each comprising a non-woven layer having a leg area, a belly area, and a waist area, said non-woven layer extending proximal to the waist opening; and
a stretch material coupled to the non-woven layer and extending about the waist opening;
wherein the non-woven layer is folded over the stretch material at the waist opening to capture the stretch material between portions of the nonwoven layer.

2. The undergarment of claim 1 wherein the stretch material includes a bi-directionally stretchable web having a width to thickness ratio of at least 3:1.

3. The undergarment of claim 1 wherein the stretch material comprises elastomeric stretch film.

4. The undergarment of claim 1 wherein the stretch material comprises at least one thread of an elastic filament.

5. The undergarment of claim 1 wherein the stretch material is attached to the non-woven by adhesives, ultrasonic bonding, heat or embossing.

6. A protective undergarment comprising:
a front portion and a back portion, each portion comprising an outer layer and an inner layer having different lengths, each of said front and back portions having:
a waist area comprising a stretchable web having a width to thickness ratio of at least 3:1;
a leg area longitudinally spaced from the waist area; and
a belly area positioned between the waist area and the leg area;
wherein the longer of the inner and outer layer of the front and back portions is folded over the shorter of the inner and the outer layer of the front and back portions at the waist end to sandwich the stretchable web therebetween.

7. The undergarment of claim 6 wherein the stretchable waist band material comprises elastomeric stretch film.

8. The undergarment of claim 6 wherein the stretchable web is attached to the front and back portions by adhesives, ultrasonic bonding, heat or embossing.

9. The undergarment of claim 6 wherein the stretchable web of the front and back portions comprises a single web of material divided into two pieces, one for each of the front and back portions.

10. The undergarment of claim 6 wherein the stretchable web of the front and back portions comprises separate elastic webs, one for each of the front and back panels.

11. The undergarment of claim 6 wherein the stretchable web of the front and back portions covers an entire circumference of the undergarment when the undergarment is assembled.

12. A protective undergarment comprising:
a front portion and a back portion each having a stretchable waist area, a stretchable leg area longitudinally spaced from the waist area, and a stretchable belly area positioned between the waist area and the leg area;
said belly area being separated from the waist area thereby defining a substantially non-stretchable portion therebetween; and
said belly area being separated from the leg area thereby defining a substantially non-stretchable portion therebetween.

13. The undergarment of claim 12 wherein the non-stretchable portions have a width in the longitudinal direction of about 0.5 cm to about 3 cm.

14. The undergarment of claim 12 wherein the non-stretchable portions have a width in the longitudinal direction of about 1 cm.

15. The undergarment of claim 12 wherein the stretchable waist areas comprise at least one elastic thread.

16. The undergarment of claim 12 wherein the stretchable waist areas comprise a stretchable film.

17. The undergarment of claim 12 wherein the stretchable belly areas comprise at least one elastic thread.

18. The undergarment of claim 12 wherein the stretchable belly areas comprise a stretchable film.

19. The undergarment of claim 12 wherein the stretchable leg areas comprises at least one elastic thread.

20. The undergarment of claim 19 wherein the stretchable leg areas are deactivated by cutting or removing a portion of the elastic thread where the crotch insert is to be attached to the front and back panels.

21. The undergarment of claim 12 wherein the stretchable leg area comprises a stretchable film.

22. The undergarment of claim 21 wherein the stretchable leg areas are deactivated by cutting or removing a portion of the stretchable film where the crotch insert is to be attached to the front and back panels.

23. The undergarment of claim 12 wherein the stretchable leg areas are deactivated where the crotch insert is to be attached to the front and back panels.

24. A method of manufacturing a protective undergarment comprising the steps of:
forming from at least one continuous web of non-woven material front and back outer portions;
coupling a first continuous web of stretch material to the front outer portion of the non-woven material to form a front waistband;
coupling a second continuous web of stretch material to the back outer portion of the non-woven material to form a rear waistband;
folding a portion of said non-woven material over the front and rear waistbands; and
connecting the front and rear waist bands to form a continuous waist opening.

25. The method of claim 24 wherein the stretch material comprises a bi-directionally stretchable web having a width to thickness ratio of at least 3:1.

26. The method of claim 24 wherein the stretch material comprises a stretchable film.

27. The method of claim 24 wherein the stretch material comprises at least one elastic thread.

28. The method of claim 24 wherein the coupling steps comprise coupling the stretch material to the non-woven by adhesives, ultrasonic bonding, heat or embossing.

29. The method of claim 24 further comprising the step of elongating the stretch material along its length prior to coupling the material to the front and back outer portions.

30. A method of manufacturing a protective undergarment comprising the steps of:
forming from at least one continuous web of non-woven material front and back outer portions;
coupling a first continuous web of stretch material having a width to thickness ratio of at least 3:1 to the front outer portion of the non-woven material to form a front waistband;
coupling a second continuous web of stretch material having a width to thickness ratio of at least 3:1 to the back outer portion of the non-woven material to form a rear waistband; and
connecting the front and rear waist bands to form a continuous waist opening.

31. The method of claim 30 further comprising the step of elongating the stretchable web along its length prior to coupling the web to the front and back outer portions.

32. The method of claim 30 further comprising the step of folding each of the front outer and back outer portions over the stretchable webs.

33. The method of claim 30 further comprising the step of adding an additional non-woven web of material over each of the stretchable webs.

34. The method of claim 30 further comprising the step of forming from a second continuous web of non-woven material front and back inner portions and sandwiching the first stretchable web between the inner front portion and the outer front portion and the second stretchable web between the inner back portion and the outer back portion.

35. The method of claim 30 wherein the step of fixing the stretchable web to each of the front and back outer portions further comprises attaching the stretchable web to the non-woven by adhesives, ultrasonics, heat or embossing.

36. The method of claim 30 further comprising the step of attaching the stretchable web to the front and back outer panels at intermittent points of attachment so that less than 100% of the stretchable web is attached to the front and back portions.

37. The method of claim 36 further comprising the step of creating repeating patterns from the points of attachment.

38. The method of claim 30 wherein the front and the back portions each are formed with a repeating wave-shaped pattern along a crotch end of the portions.

39. The method of claim 38 further comprising the step of indexing the front and back outer portions to align a peak of a wave of the wave-shaped pattern with a peak of an opposing wave of the wave-shaped pattern.
